(19) United States
(12) Patent Application Publication
(10) Pub. No.: US 2026/0060789 A1
(43) Pub. Date: Mar. 5, 2026

(54) PROCESS FOR THE PREPARATION OF A ZIRCONIA BLANK

(71) Applicant: Ivoclar Vivadent AG, Schaan (LI)

(72) Inventors: Bo Jiang, Werdenberg (CH); Christian Ritzberger, Grabs (CH); Frank Rothbrust, Röns (AT)

(21) Appl. No.: 19/384,552

(22) Filed: Nov. 10, 2025

Related U.S. Application Data
(63) Continuation of application No. 18/459,775, filed on Sep. 1, 2023.
Application No. 18/459,775 is a division of application No. 16/674,047, filed on Nov. 5, 2019, now abandoned.

(30) Foreign Application Priority Data
Nov. 29, 2018 (EP) 18209272.6

(51) Int. Cl. A61C 13/00; A61K 6/818; B28B 1/26; C04B 35/486; C04B 35/64

(52) U.S. Cl. CPC A61C 13/0022; A61C 13/0006; A61K 6/818; B28B 1/26; C04B 35/486; C04B 35/64; C04B 2235/3246; C04B 2235/5445; C04B 2235/5454; C04B 2235/6027; C04B 2235/606; C04B 2235/608; C04B 2235/6567; C04B 2235/661

(57) ABSTRACT

A zirconia blank produced by introducing a zirconia suspension into a porous mould and demoulding the blank formed as well as the use of the optionally presintered blank formed for the preparation of a dental restoration using a very short dense-sintering process.

### CROSS-REFERENCE TO RELATED APPLICATIONS

This application is a continuation application of and claims priority to U.S. application Ser. No. 18/459,775, filed on Sep. 1, 2023, which is a divisional application of and claims priority to U.S. application Ser. No. 16/674,047, filed on Nov. 5, 2019, which claims priority to European Patent Application No. 18209272.6 filed on Nov. 29, 2018, the disclosures of which are incorporated herein by reference in their entirety.

### TECHNICAL FIELD

The present invention relates to a process for the preparation of a zirconia blank which, after desired shaping, can be densely sintered within a very short time to form dental restorations with outstanding optical properties.

Zirconia ceramics are often used for the production of fully anatomical dental restorations. They offer high clinical safety, are metal-free, can also be used in minimally invasive preparations and are very attractive in terms of price compared with other metal-free restorations. The restorations are usually milled or ground out of presintered blanks, densely sintered by thermal treatment and finally optionally provided with a veneer and/or glazed.

Processes for the preparation of zirconia blanks and the processing thereof to form dental restorations are known.

WO 2014/209626 and corresponding U.S. Ser. No. 10/004,668, which is hereby incorporated by reference, describe a process for the production of zirconia blanks which, after presintering and machining by means of CAD/CAM processes, can be processed by dense sintering at temperatures of less than 1200° C. to form dental restorations. The restorations are characterized in that they are not only translucent but also opalescent. For the production of the blanks suspensions of nanoscale zirconia particles with an average primary particle size of not more than 20 nm are for example poured into corresponding moulds. However, the preparation of the suspensions is laborious as the commercially available suspensions used as raw material first require a lengthy concentration step. The green compacts obtained by the casting also need to be dried very slowly, which takes several days, specifically in the case of blanks with a greater thickness, to avoid the formation of cracks. Finally, the whole dense-sintering procedure is also very time-consuming and takes more than 4 hours, whereby the patient's desire cannot be fulfilled that the restoration ground and milled out of the blank is completed quickly.

WO 2009/061410 and corresponding US 2009115084, which is hereby incorporated by reference, and WO 2013/181018 and corresponding U.S. Pat. No. 9,790,129, which is hereby incorporated by reference, describe processes for the preparation of zirconia blanks by slip casting of suspensions. The blanks are said to produce translucent dental restorations after drying, presintering, machining, e.g. by CAD/CAM processes, and subsequent dense sintering. However, no specific examples of the process and the suspension used therein are given, nor are the precise conditions for the dense sintering and in particular its duration specified.

EP 826 642 and corresponding U.S. Pat. No. 5,975,905, which is hereby incorporated by reference, describe a process for the production of a ceramic framework for a dental restoration. The process includes the slip casting of a suspension with an alumina and zirconia particle content to form a ceramic film, layering the film onto a gypsum model of a tooth, exerting pressure to join the film to the model, heat treatment at 500° C., sintering at 1150° C., coating the sintered body with glass powder and further heating.

KR 101416654 B1 describes a process for the production of ZrO₂ blocks using zirconia waste, such as accumulates in particular in dental laboratories. From this waste, after debinding and grinding, aqueous zirconia suspensions are produced, from which bodies with the desired shape are produced by slip casting. These bodies are cold-isostatically pressed and then sintered twice.

WO 2018/049331 and corresponding US 2018072628, which is hereby incorporated by reference, describe dental zirconia blanks for CAD/CAM applications which, due to the use of different ZrO₂ particles, exhibit a gradient of properties and in particular a colour gradient. The blanks can be formed by slip casting mixtures of suspensions with differently coloured and differently sized zirconia particles, wherein use is to be made of the different settling behaviour of the particles.

However, the conventional processes for the production of zirconia blanks are laborious and the blanks produced with them require long processing times for the dense sintering, in particular in order to achieve the desired optical properties, such as a high translucence. The whole process of dense-sintering conventional blanks, starting with the heating up to the sintering temperature and ending with the cooling down to room temperature, typically takes considerably more than 4 hours, which contributes significantly to an unsatisfactorily long duration for the processing to form dental restorations.

### SUMMARY

The object of the invention is therefore to provide a process for the preparation of a zirconia blank which avoids the disadvantages of the conventional processes and makes it possible to produce blanks in a short time which can be processed to form dental restorations with very good optical and mechanical properties.

### DETAILED DESCRIPTION

This object is achieved according to the invention by the process for the preparation of a zirconia blank according to claims 1 to 16. A subject of the invention is also the zirconia blank according to claims 17 to 19, the use of the zirconia blank according to claim 20 as well as the process for the preparation of a dental restoration according to claims 21 to 24.

The process according to the invention for the preparation of a zirconia blank is characterized in that
(a) a suspension of zirconia in a liquid medium is introduced into a mould having pores,
(b) the liquid medium is at least partially removed via the pores and
(c) the blank formed is removed from the mould,

wherein the suspension has a zirconia content of from 68 to 88 wt.-%, preferably 70 to 86 wt.-% and particularly preferably 75 to 85 wt.-%.

Surprisingly, it has been found that, after corresponding shaping, a blank obtainable by means of the process according to the invention can be densely sintered in only a very short time to form dental restorations which nevertheless have very good optical properties and in particular a high translucence, and thus also meet the high demands on dental restorations from an aesthetic point of view. It is thus possible to produce for a patient a dental restoration from a blank that is both shaped as desired and densely sintered and to fit it for him within only one session at the dentist's. Such a quick provision of a dental restoration is also referred to as “chairside” treatment and it is naturally extremely attractive for the patient. The process according to the invention is thus greatly superior to conventional processes, in which very long sintering times have to be tolerated just to achieve a satisfactory translucence.

The zirconia in the suspension has in particular a particle size of from 50 to 250 nm, preferably from 60 to 250 nm and particularly preferably 80 to 250 nm, measured as the d₅₀ value, relative to the volume of the particles. The particle size is determined in particular with the static laser scattering (SLS) process according to ISO 13320:2009, e.g. using an LA-960 particle analyzer from Horiba, or the dynamic light scattering (DLS) process according to ISO 22412:2017, e.g. using a NANO-flex particle measurement device from Colloid Metrix.

The primary particle size of the zirconia lies in particular in the range of from 30 to 100 nm and it is usually also determined with a dynamic light scattering (DLS) process as described above or by means of scanning electron microscopy.

The zirconia is, in particular, zirconia based on tetragonal zirconia polycrystal (TZP). Zirconia is preferred which is stabilized with Y₂O₃, La₂O₃, CeO₂, MgO and/or CaO and in particular is stabilized with 2 to 14 mol-%, preferably with 2 to 10 mol-% and particularly preferably 2 to 8 mol-% of these oxides, relative to the zirconia content.

The zirconia used in the process according to the invention can also be coloured. The desired colouring is achieved in particular by adding one or more colouring elements to the zirconia. The addition of colouring elements is sometimes also referred to as doping and is usually effected during the production of the zirconia powder by coprecipitation and subsequent calcination. Examples of suitable colouring elements are Fe, Mn, Cr, Ni, Co, Pr, Ce, Eu, Gd, Nd, Yb, Tb, Er and Bi.

The zirconia in the suspension can also be a mixture of zirconia powders with different composition, leading in particular to a different colour and/or translucence in the finally produced dental restoration. With the aid of a mixture of differently coloured zirconia powders, the desired colour for the dental restoration produced from the blank can thus be set simply and in a targeted manner. In the same way, the translucence of the dental restoration produced can also be adjusted in a targeted manner by using a mixture of zirconia powders of different translucence. The degree of translucence of the dental restoration produced can in particular be controlled by the yttrium oxide content of the zirconia powders used.

The suspension can also be a mixture of different suspensions, for example with differently coloured zirconia.

In the process according to the invention the zirconia is present as a suspension in a liquid medium. This liquid medium contains in particular water.

It is moreover preferred that the liquid medium has only small amounts of organic components and it therefore contains organic components in an amount of in particular not more than 5 wt.-%, preferably not more than 3 wt.-%, further preferably not more than 2 wt.-% and particularly preferably not more than 1 wt.-%, relative to the amount of solid in the suspension.

In a further preferred embodiment the liquid medium contains organic components in an amount of from 0.05 to 5 wt.-%, in particular 0.1 to 3 wt.-%, particularly preferably 0.1 to 2 wt.-% and particularly preferably 0.1 to 1 wt.-%, relative to the amount of solid in the suspension.

In particular, dispersants, binders, agents for adjusting the pH value, stabilizers and/or defoamers come into consideration as organic components.

The dispersant serves to prevent the agglomeration of suspended particles to form larger particles. The amount of dispersant in the liquid medium is in particular 0.01 to 5 wt.-%, preferably 0.1 to 2 wt.-% and particularly preferably 0.1 to 1 wt.-%, relative to the amount of solid in the suspension.

Suitable dispersants are water-soluble polymers, such as polyvinyl alcohols, polyethyleneimines, polyacrylamides, polyethylene oxides, polyethylene glycols, homo- and copolymers of (meth)acrylic acid, polyvinylpyrrolidone, biopolymers, such as starches, alginates, gelatins, cellulose ethers, such as carboxymethylcellulose, vinylsulfonic acid and vinylphosphonic acid.

Preferred dispersants are amino alcohols, such as ethanolamine; glycols, such as ethylene glycol and dipropylene glycol; carboxylic acids, such as maleic acid and citric acid, and carboxylic acid salts; as well as mixtures of these dispersants.

It is further preferred that the liquid medium contains at least one compound selected from amino alcohol, glycol, carboxylic acid and carboxylic acid salt. The liquid medium particularly preferably contains at least one compound selected from ethanolamine, ethylene glycol, dipropylene glycol, citric acid and citric acid salt.

The binder promotes the cohesion of particles in the blank present after step (c). The amount of binder in the liquid medium is in particular 0.01 to 5 wt.-%, preferably 0.01 to 3 wt.-% and particularly preferably 0.01 to 2 wt.-%, relative to the amount of solid in the suspension.

Examples of suitable binders are methyl cellulose, sodium carboxymethylcellulose, starches, dextrins, sodium alginate, ammonium alginate, polyethylene glycols, polyvinyl butyral, acrylate polymers, polyethyleneimine, polyvinyl alcohol and polyvinylpyrrolidone.

Preferred binders are polyvinyl alcohol, polyvinyl acetate, polyvinylpyrrolidone, polyacrylic acid, copolymers of acrylic acid esters and acrylic acid, polyethyl acrylate, polymethacrylic acid, polymethyl methacrylate, ammonium polyacrylate, ammonium polymethacrylate, polyethylene glycol and solid copolymers of ethylene glycol and propylene glycol.

It is a further advantage of the blank obtainable by the process according to the invention that, despite small amounts of binder, it has sufficient strength even without prior presintering to be able to be processed to form the desired dental restoration by machining, e.g. by means of grinding and milling.

Acids and bases come into consideration in particular as agents for adjusting the pH value and as stabilizers, such as carboxylic acids, e.g. 2-(2-methoxyethoxy)acetic acid and 2-[2-(2-methoxyethoxy)ethoxy]acetic acid, inorganic acids, e.g. hydrochloric acid and nitric acid, as well as ammonium hydroxide and tetramethylammonium hydroxide. It is preferred that the liquid medium contains tetramethylammonium hydroxide.

The defoamer serves to prevent air bubbles in the suspension. It is typically used in the liquid medium in an amount of from 0.001 to 1 wt.-%, preferably 0.001 to 0.5 wt.-% and particularly preferably 0.001 to 0.1 wt.-%, relative to the amount of solid in the suspension. Examples of suitable defoamers are paraffin, silicone oils, alkyl polysiloxanes, higher alcohols, propylene glycol, ethylene oxide-propylene oxide adducts and in particular alkyl polyalkylene glycol ether.

It is possible that components of the liquid medium perform several functions and are e.g. both dispersant and binder or both dispersant and agent for adjusting the pH value as well as stabilizer.

Because of the small proportion of organic components, they can also be burned out of the blank within a short time. This procedure is usually also referred to as debinding.

It is further preferred that the suspension has a viscosity of from 5 mPas to 500 mPas, preferably 5 mPas to 400 mPas and particularly preferably 5 to 300 mPas. The viscosity was measured with a rotational viscometer with a cone-plate system, diameter 50 mm and angle 1° (MCR302 Modular Compact Rheometer, from Anton Paar GmbH), at a shear rate in the range of from 0.1 to 1000 s⁻¹ and a temperature of 25° C.

To produce the suspension, the zirconia is typically thoroughly mixed in powder form with the liquid medium. Mixtures of, for example, differently coloured zirconia can also be used here. During this mixing, any agglomerates present are usually also broken up and grinding of the zirconia used can also be effected in order to produce the desired particle size. The mixing of zirconia and liquid medium can therefore advantageously be carried out for example in agitator bead mills.

The suspension is then in step (a) of the process according to the invention introduced into a mould which has pores. The introduction is usually effected by pouring. The pores are openings through which the liquid medium can at least partially be removed from the suspension in step (b), with the result that zirconia particles can be deposited in the mould and ultimately can form the blank. The geometry of the mould thus corresponds to the geometry of the desired blank. The mould can therefore also already have, for example, a holding device formed in one piece with the blank, such as a holding pin, which makes the later attachment thereof, for example by gluing, superfluous.

In a preferred embodiment of the process according to the invention, the blank formed in step (c) therefore has a holding device.

Typically, substantially all of the liquid medium is removed via the pores. However, it is also possible that remaining liquid not removed via the pores is poured or suctioned out of the mould. This is usually the case when a sufficiently thick layer of zirconia particles has already been deposited.

To produce blanks which have areas with different colour and/or translucence, suspensions of zirconia powders with different compositions can be introduced into the mould one after another and layers of different zirconia particles with in each case a desired thickness can be deposited.

In a preferred embodiment of the process according to the invention, therefore, in step (a) suspensions of zirconia powders with different compositions and in particular with different colour and/or translucence are introduced into the mould one after another. Here, measures are typically taken that the suspensions with different compositions do not intermix. This can be achieved, e.g., in that the layer deposited from a suspension is first dried and thus solidified before a further suspension with a different composition is introduced into the mould.

The mould can for example be one of the moulds usually used for slip casting or pressure casting processes. These are in particular moulds with a wall made of gypsum, through which, because of the capillary action of the gypsum pores, water can be removed from the suspension. However, moulds made of plastic, ceramic or metal which already have pores or in which pores are provided, e.g. by providing them with filter elements, such as membrane filters, paper filters and sintered filters, can also be used.

The mould used is in particular comprised of several parts in order to facilitate the simple removal of the blank formed from the mould. In a particularly preferred embodiment the mould has connections via which pressure, for example by means of compressed air, can be exerted on the introduced suspension and/or a negative pressure can be applied to the pores. Both measures serve to speed up the removal of the liquid medium from the mould and thus to shorten the process. With the help thereof, a very quick and thus economical production of zirconia blanks is possible, which is particularly advantageous in particular in the case of manufacturing on an industrial scale.

The blank formed by means of the process according to the invention can have any desired shape. Here, in particular a shape is chosen which allows simple machining of the blank in usual dental grinding and milling devices. The blank is preferably present as a block, a block with an interface, such as e.g. a hole as implant interface, a disc or a toothlike preform. It is preferred here that the blank also has a holding device, such as a holding pin, which is formed in one piece with the blank. A previously usual later attachment of a holder by gluing is thereby made superfluous. The holding device serves to fix the blank in a machining device, such as a CAM machine.

After the removal from the mould in step (c) the blank formed is usually dried. The drying is effected in particular at a temperature of from 20 to 60° C., preferably 20 to 50° C. and particularly preferably 20 to 40° C. The duration of the drying is in particular 0.1 to 24 h, preferably 0.5 to 12 h and particularly preferably 1 to 6 h. The drying can be effected e.g. in a climate control cabinet or by microwave drying.

The blank obtained after the completion of step (c) and optionally subsequent drying is present in the so-called green state and it is therefore also referred to as a green compact. Surprisingly, in this state the blank has a very high density and it is preferred that it has a density of from 3.3 to 4.0 g/cm³, in particular 3.35 to 3.9 g/cm³ and preferably 3.4 to 3.9 g/cm³. The density was determined by means of mercury porosimetry according to ISO 15901-1:2016.

In a further preferred embodiment this blank present in the green state has a pore volume of from 0.08 to 0.14 cm³/g, in particular 0.08 to 0.12 cm³/g and particularly preferably 0.08 to 0.10 cm³/g. The pore volume was determined by means of mercury porosimetry according to ISO 15901-1:2016.

In another preferred embodiment this blank present in the green state has a pore diameter of from 0.02 to 0.12 μm, in particular 0.03 to 0.10 μm and particularly preferably 0.04 to 0.08 μm, measured as the D₅₀ value relative to the volume of the particles. The pore diameter was determined by means of mercury porosimetry according to ISO 15901-1:2016.

The blank can already be machined in this green state, optionally after prior removal of organic components, in order to give it the shape of the desired dental restoration.

The removal of organic components is also referred to as debinding and is effected in particular by heat treatment at a temperature of about 600° C. A heating rate of 0.125 K/min to 10 K/min, preferably 0.250 K/min to 5 K/min and particularly preferably 0.5 K/min to 2 K/min starting from room temperature to about 600° C. is usually chosen.

However, it is preferred that the blank in the green state is also presintered. Through the presintering, the strength and hardness of the blank are increased. The presintering is effected in particular at a temperature in the range of from 600 to 1100° C., preferably at 700 to 1050° C. and particularly preferably at 800 to 1000° C. for a duration of in particular 5 min to 24 h, preferably 10 min to 12 h and particularly preferably 30 min to 6 h.

It is preferred that the presintered blank has a density of from 3.4 to 4.0 g/cm³, in particular 3.5 to 4.0 g/cm³ and particularly preferably 3.6 to 3.9 g/cm³.

According to a further preferred embodiment the presintered blank has a pore volume of from 0.08 to 0.14 cm³/g, in particular 0.08 to 0.12 cm³/g and preferably 0.09 to 0.11 cm³/g. The pore volume was determined by means of mercury porosimetry according to ISO 15901-1:2016.

In a further preferred embodiment the presintered blank has a maximum pore diameter of less than 0.15 μm, preferably less than 0.12 μm and particularly preferably less than 0.08 μm. The maximum pore diameter was determined by means of mercury porosimetry according to ISO 15901-1:2016.

The invention is also directed to a zirconia blank which is obtainable by the process according to the invention. This blank evidently has a special structure compared with conventional blanks as, after desired shaping, it can be densely sintered within a very short time to form a dental restoration with very good optical properties, such as translucence.

The invention is further directed to a zirconia blank which is presintered and has a density of from 3.4 to 4.0 g/cm³, in particular 3.5 to 4.0 g/cm³ and preferably 3.6 to 3.9 g/cm³ and/or a pore volume of from 0.08 to 0.14 cm³/g, in particular 0.08 to 0.12 cm³/g and preferably 0.09 to 0.11 cm³/g and/or a maximum pore diameter of less than 0.15 μm, in particular less than 0.12 μm and preferably less than 0.08 μm.

The blanks according to the invention can be used advantageously to manufacture dental restorations from them. After the shaping to form the desired dental restoration, they can be densely sintered to form dental restorations with outstanding optical properties and high strength in only a very short time, which makes it possible to provide a patient with a dental restoration very quickly.

The blanks according to the invention are moreover characterized in that they experience only low linear shrinkage during the dense sintering. The preparation of dental restorations with precisely the desired dimensions is thereby made easier and the accuracy of fit thereof is improved.

It is preferred that the blanks according to the invention have a linear shrinkage of less than 18%, in particular less than 17% and particularly preferably less than 16%. The linear shrinkage S results from the following formula and, for the determination thereof, measurements were carried out by means of a Netzsch DIL402 Supreme dilatometer in a temperature range of from 20° C. to 1550° C. at a heating rate of 2 K/min on test pieces with the dimensions length=25 mm±1 mm, width=5 mm±0.5 mm and height=4 mm+0.5 mm.

$$Linear shrinkage : S = \frac{Length raw - Length sintered}{Length raw} * 100 in \%$$

From the linear shrinkage S, the volume shrinkage S_{Vol} can be calculated according to the following formula:

$$Volume shrinkage : {S}_{Vol} = [ 1 - {( 1 - \frac{Linear shrinkage}{100} )}^{3} ] * 100 in \%$$

The invention is therefore also directed to the use of the blanks according to the invention for the preparation of a dental restoration. Preferred embodiments of this use are explained in the following in connection with the description of the process according to the invention for the preparation of a dental restoration.

The process according to the invention for the preparation of a dental restoration is characterized in that a blank according to the invention is given the shape of the dental restoration and the blank is densely sintered at a sintering temperature of from 1200 to 1600° C., in particular 1300 to 1550° C. and preferably 1350 to 1500° C.

The shaping of the blank is effected in particular by machining, e.g. by means of computer-controlled grinding and milling devices, such as are usual in the dental field.

Following the shaping, colouring solution can optionally also be applied to the blank in order to achieve the desired colour in the finally obtained dental restoration.

The subsequent dense sintering leads to the blank shaped to form the dental restoration having a density of in particular more than 5.9 g/cm³, preferably more than 6.00 g/cm³ and particularly preferably more than 6.02 g/cm³.

Also in view of this high density, the dental restoration obtained has outstanding mechanical properties.

It is a particular advantage that the whole dense-sintering process with the heating from room temperature, the holding at the maximum sintering temperature and the cooling takes only a very short period of time and, after the completion thereof, dental restorations with the sought high translucence and very good mechanical properties are nevertheless obtained. The process according to the invention is thus superior to conventional processes, which require a very long period of time in order to produce a dental restoration with comparable translucence by sintering. The process according to the invention thus combines the advantage of a very short process duration with that of the very good optical and mechanical properties of the dental restoration produced.

In a preferred embodiment of the process according to the invention, the period for heating the shaped blank from room temperature to the sintering temperature for the dense sintering, holding at the sintering temperature and cooling to the final temperature is not more than 120 min, in particular not more than 60 min, preferably not more than 40 min and particularly preferably not more than 30 min. By “final temperature” is meant here a temperature at which the sample can be picked up by hand, and it is in particular 15 to 80° C., preferably 25 to 60° C. and particularly preferably about 50° C. By “room temperature” is meant a temperature of in particular 15 to 30° C., preferably 20 to 25° C. and particularly preferably about 25° C.

The heating rate is in particular more than 50 K/min, preferably more than 100 K/min and particularly preferably more than 200 K/min. The holding time is in particular less than 30 min, preferably less than 20 min and particularly preferably less than 10 min. The cooling rate from the sintering temperature to the final temperature is in particular more than 50 K/min, preferably more than 100 K/min and particularly preferably more than 150 K/min.

Through the low linear shrinkage which the shaped blank according to the invention experiences during the dense sintering, the preparation of dental restorations with precisely the desired dimensions is made easier and the accuracy of fit thereof is improved.

In a preferred embodiment, the blank is
(a) subjected to a first heat treatment,
(b) subjected to a second heat treatment and
(c) cooled,

wherein the heat treatment in step (a) is effected at a lower pressure than the heat treatment in step (b). In step (a) the blank is preferably heated to a temperature which lies in the range of from 1100 to 1600° C., in particular in the range of from 1200 to 1500° C., preferably in the range of from 1250 to 1450° C. and further preferably in the range of from 1300 to 1400° C. and is most preferably about 1350° C. It is further preferred that the heat treatment in step (a) is effected at a pressure of less than 200 mbar, preferably less than 100 mbar and particularly preferably less than 50 mbar, and in particular at a pressure in the range of from 0.1 to 200 mbar, preferably 1 to 150 mbar and particularly preferably 50 to 100 mbar. In step (b) the blank is preferably (b1) optionally further heated and (b2) held and sintered at a, preferably constant, temperature in the range of from 1100 to 1700° C., in particular in the range of from 1300 to 1600° C. and preferably in the range of from 1350 to 1550° C. and particularly preferably at a temperature of about 1500° C. It is also preferred that the heat treatment in step (b) is effected at a pressure of more than 500 mbar and in particular at ambient pressure and in particular is effected in an oxygen-containing atmosphere such as air, oxygen-enriched air or oxygen. It is particularly preferred that an oxygen-containing atmosphere, preferably air, oxygen-enriched air or oxygen, flows through the heating chamber used for the heating during step (b) discontinuously or preferably continuously, in particular at a flow rate of from 0.1 to 50 l/min, preferably 1 to 20 l/min and particularly preferably 4 to 8 l/min. In a particularly preferred embodiment, in step (a) the blank is heated to a temperature which lies 0 to 500 K, in particular 10 to 250 K, preferably 50 to 200 K and particularly preferably 100 to 150 K below the temperature or the temperature range at or in which the blank is held in step (b).

The dental restoration obtained after the dense sintering can optionally also be provided with a veneer, polished and/or glazed.

The dental restoration produced using the process according to the invention is in particular a bridge, an inlay, an onlay, a crown, a veneer, an implant, a facet or an abutment.

The invention is explained in more detail in the following with reference to examples.

### EXAMPLES

### Example 1—Suspension with 76 wt.-% Zirconia

3.15 g of a dispersant containing citric acid or citric acid salt (Dolapix CE64, from Zschimmer & Schwarz) and 1.5 g tetramethylammonium hydroxide were dissolved one after the other in 194.4 g distilled water. The solution had a pH value of 10-10.5.

This solution was placed in the storage tank of a MicroCer agitator bead mill (from Netzsch), the grinding chamber and rotor of which was made of zirconia. The grinding chamber was filled with 60 ml zirconia grinding beads with a diameter of 0.2-0.3 mm (from Tosoh). At a rotational speed of the rotor of 1500 rpm the solution was pumped continuously through the grinding chamber using a peristaltic pump (tube internal diameter 8 mm). 630 g zirconia powder which was partially stabilized with 3 mol-% Y₂O₃(TZ-PX-245 from TOSOH Corporation, primary particle size: 40 nm) were then, continuously and with stirring, added to the solution in the storage tank. Once the addition of the zirconia powder was completed, the mixture obtained was pumped through the grinding chamber and back into the storage tank continuously for 45 min at a rate of about 40 l/h. The suspension prepared in this way was transferred into a plastic measuring beaker and stirred very slowly by means of a magnetic stirrer in order to remove trapped air bubbles. In addition, one drop of an alkyl polyalkylene glycol ether was added as defoamer (Contraspum, from Zschimmer & Schwarz). The suspension obtained had a zirconia content of 76 wt.-%. The viscosity η of the suspension was 7.25 mPas (at a shear rate of 500 s⁻¹ and a temperature of 25° C.).

### (A) Preparation of Test Pieces

Test pieces were produced, on which different properties were determined. For the preparation thereof, the suspension obtained was poured into a disc-shaped mould which consisted of a plate made of porous gypsum with a plastic ring secured thereon. Through the capillary action of the porous gypsum, water was removed from the suspension, and a monolithic green body formed. The green body was left in the mould and dried in air for 24 h. After completion of the drying the test piece had released itself from the mould.

The respective test pieces had the following properties:
Linear shrinkage: 15.43% (test piece: length=25 mm±1 mm, width=5 mm±0.5 mm and height=4 mm±0.5 mm)
Density: 3.678 g/cm³ (test piece: length=5 mm±1 mm, width=5 mm±1 mm and height=10 mm±1 mm)

### (B) Preparation of Blocks for a “Chairside” Application

A pressure casting mould was used for the preparation of blocks for a “chairside” application. This pressure casting mould consisted of a top part with a connection for compressed air (positive pressure 0.1 to 100 bar), a middle part which corresponded to the desired shape of the block, and a bottom part which was provided with a membrane filter, paper filter and sintered filter, and on the base had a connection for applying a vacuum. The membrane filter had a pore size of 200 nm.

The suspension was poured into the pressure casting mould. Then compressed air with a pressure of 6 bar was supplied via the top part and a vacuum of about 0.05 bar was applied via the bottom part. After 4 h, the addition of compressed air and the application of a vacuum was ended. The pressure casting mould was disassembled and the block obtained was dried in air at 20° C. for 24 h.

The blocks produced in this way were presintered at 950° C. for 2 h. The heating rate was 0.250 K/min. The blocks were then cooled to room temperature within 24 h. The hardness of the blocks was measured by means of the Vickers method according to ISO 14705:2008 and EN ISO 6507-1:2005 at a load of 2.5 kg. The hardness was 992.95 N/mm².

A front tooth crown to which colouring liquid had been applied by means of a brush for staining was dry milled from the presintered blocks by means of a CAD/CAM machine (Zenotec® Select from Ivoclar Vivadent AG). After the colouring, the crown was dried and densely sintered in a sintering furnace (Programat CS4, program 1, from Ivoclar Vivadent AG). The sintering took only about 35 min, as the temperature schedule below shows.

| | |
|---|---|
| About 25° C. to 900° C. | (6.7 min) |
| 900° C. to 1460° C. | (11.2 min) |
| 1460° C. to 1460° C. | (5.0 min) |
| 1460° C. to 1200° C. | (3.7 min) |
| 1200° C. to about 1000-950° C. | (45-50 s) (furnace opens) |
| 950° C.-1000° C. to about 50° C. | (7 min) |

### Example 2—Suspension with 83 wt.-% Zirconia

For the preparation of a suspension with 83 wt.-% zirconia and the processing thereof to form test pieces (A) and blocks for “chairside” application (B), Example 1 was repeated with the variation that the solution contained 164.5 g distilled water, 4.05 g dispersant containing citric acid or citric acid salt (Dolapix CE64, from Zschimmer & Schwarz) and 1.5 g tetramethylammonium hydroxide, and that 810 g zirconia powder which was partially stabilized with 5 mol-% Y₂O₃(TZ-PX-430 from TOSOH Corporation, primary particle size: 90 nm) were added and the presintering was carried out at 900° C. for 2 h, wherein the heating rate was likewise 0.250 K/min.

The viscosity η of the suspension was 7.0 mPas (at a shear rate of 1000 s⁻¹ and a temperature of 25° C.).

The respective test pieces obtained in accordance with (A) had the following properties:
Linear shrinkage: 14.43% (test piece: length=25 mm±1 mm, width=5 mm±0.5 mm and height=4 mm±0.5 mm)
Density: 3.791 g/cm³ (test piece: length=5 mm±1 mm, width=5 mm±1 mm and height=10 mm±1 mm)

The hardness of the presintered blocks obtained in accordance with (B) was measured by means of the Vickers method according to ISO 14705:2008 and EN ISO 6507-1:2005 at a load of 2.5 kg. The hardness was 496.91 N/mm².

A front tooth crown to which colouring liquid had been applied by means of a brush for staining was dry milled from the presintered blocks by means of a CAD/CAM machine (Zenotec® Select from Ivoclar Vivadent AG). After the colouring, the crown was dried and densely sintered in a sintering furnace (Programat CS4, program 1, from Ivoclar Vivadent AG). The sintering took only 34 min, as the temperature schedule below shows.

| | |
|---|---|
| About 25° C. to 900° C. | (6.7 min) |
| 900° C. to 1460° C. | (11.2 min) |
| 1460° C. to 1460° C. | (5.0 min) |
| 1460° C. to 1200° C. | (3.7 min) |
| 1200° C. to about 1000-950° C. | (45-50 s) (furnace opens) |
| 950° C.-1000° C. to about 50° C. | (7 min) |

### Example 3—Suspension with 80 wt.-% Zirconia

For the preparation of a suspension with 80 wt.-% zirconia and the processing thereof to form test pieces (A), Example 1 was repeated with the variation that the solution contained 179.5 g distilled water, 3.6 g dispersant containing citric acid or citric acid salt (Dolapix CE64, from Zschimmer & Schwarz) and 1.5 g tetramethylammonium hydroxide, and that 720 g zirconia powder which was partially stabilized with 4.25 mol-% Y₂O₃(TZ-PX-551 from TOSOH Corporation, primary particle size: 90 nm) were added.

The viscosity η of the suspension was 14.6 mPas (at a shear rate of 500 s⁻¹ and a temperature of 25° C.).

The test pieces obtained according to (A) had the following properties:
Linear shrinkage: 14.59%
Density: 3.780 g/cm³.

The test pieces obtained according to (A) were presintered at 1000° C. for 2 h. The heating rate was 0.250 K/min. The test pieces were then cooled to room temperature within 24 h. The presintered test pieces had a linear shrinkage of 13.88%.

### Example 4—Suspension with 76 wt.-% Zirconia

For the preparation of a suspension with 76 wt.-% zirconia and the processing thereof to form blocks for “chairside” application (B), Example 1 was repeated with the variation that 630 g zirconia powder which was partially stabilized with 3 mol-% Y₂O₃(AuerDent 3Y-5A A2 from Treibacher Industrie AG, primary particle size: 40 nm) were added to the solution, and that the presintering was carried out at 820° C. for 2 h, wherein the heating rate was likewise 0.250 K/min.

The hardness of the presintered blocks obtained according to (B) was measured by means of the Vickers method according to ISO 14705:2008 and EN ISO 6507-1:2005 at a load of 2.5 kg. The hardness was 440.38 N/mm².

Furthermore, a disc was sawn off the presintered blocks and densely sintered in a sintering furnace (Programat CS4 from Ivoclar Vivadent AG) within only 34 min according to the following temperature schedule:

| | |
|---|---|
| About 25° C. to 900° C. | (6.7 min) |
| 900° C. to 1460° C. | (11.2 min) |
| 1460° C. to 1460° C. | (5.0 min) |
| 1460° C. to 1200° C. | (3.7 min) |
| 1200° C. to about 1000-950° C. | (45-50 s) (furnace opens) |
| 950° C.-1000° C. to about 50° C. | (7 min) |

The densely sintered discs had a contrast ratio CR of 89.16. The contrast ratio was determined in accordance with BS 5612 (British Standard) using a spectrophotometer (Minolta CM-3700d). It is a measure of the opacity of a material, wherein a contrast ratio of 100 corresponds to a completely opaque material and a contrast ratio of 0 corresponds to a completely translucent material.

The measured value of 89.16 shows the high translucence of the samples produced according to the invention and in the case of conventionally produced blanks it can only be achieved with very long sintering times of more than 2 hours.

### Example 5—Suspension with 83 wt.-% Zirconia

For the preparation of a suspension with 83 wt.-% zirconia and the processing thereof to form test pieces (A), Example 1 was repeated with the variation that the solution contained 164.5 g distilled water, 3.15 g dispersant containing citric acid or citric acid salt (Dolapix CE64, from Zschimmer & Schwarz) and 1.5 g tetramethylammonium hydroxide, and that 810 g zirconia powder which was partially stabilized with 3 mol-% Y₂O₃ (TZ-PX-245 from TOSOH Corporation, primary particle size: 40 nm) were added.

The test pieces obtained according to (A) were debound at 500° C. and then had the following properties:
Pore volume: 0.11391 cm³/g
Pore radius: 0.0190 μm
Density: 3.5617 g/cm³ (test piece: length=5 mm±1 mm, width=5 mm±1 mm and height=10 mm±1 mm) The debound test pieces were then presintered at 1050° C. for 2 h.

The presintered blanks obtained then had the following properties:
Pore volume: 0.09931 cm³/g
Pore radius: 0.0242 μm
Density: 3.8866 g/cm³ (test piece: length=5 mm±1 mm, width=5 mm±1 mm and height=10 mm±1 mm)

Debound test pieces were likewise presintered at 1000° C. for 2 h. The presintered blanks obtained were densely sintered in a sintering furnace (Cerec Speedfire from Dentsply Sirona) within only 20 min according to the following temperature schedule:

| | |
|---|---|
| About 25° C. to 500° C. | (88 s) |
| 500° C. to 1580° C. | (190 s) |
| 1580° C. to 1580° C. | (360 s) |
| 1580° C. to 950° C. | (120 s) |
| 950° C. to about 50° C. | (420 s) |

### Example 6—Suspension with 83 wt.-% Zirconia+Vacuum

For the preparation of a suspension with 83 wt.-% zirconia and the processing thereof to form test pieces (A), Example 1 was repeated with the variation that the solution contained 164.5 g distilled water, 4.05 g dispersant containing citric acid or citric acid salt (Dolapix CE64, from Zschimmer & Schwarz) and 1.5 g tetramethylammonium hydroxide, and that 810 g zirconia powder which was partially stabilized with 3 mol-% Y₂O₃(TZ-PX-245 from TOSOH Corporation, primary particle size: 40 nm) were added.

The test pieces obtained according to (A) were debound and presintered in that they were heated to a temperature of 1000° C. at a rate of 0.25 K/min and held at this temperature for 2 h.

The presintered blanks obtained were densely sintered in a sintering furnace with MoSi₂ heating elements within a maximum of only 36 min according to the following temperature schedule, wherein the first two steps were carried out under vacuum at a pressure in the range of from about 50 to 100 mbar, and, after feeding air in, the further steps were carried out under ambient atmosphere and with continuous flushing with air.

| | | |
|---|---|---|
| About 25° C. to 950° C. | (7.1 min) | under vacuum |
| 950° C. to 1450° C. | (10 min) | under vacuum |
| 1450° C. to 1500° C. | (1 min) | continuous flow of air |
| | | at ambient atmosphere |
| 1500° C. to 1500° C. | (5 min) | continuous flow of air |
| | | at ambient atmosphere |
| 1500° C. to 1100° C. | (2.9 min) | ambient atmosphere |
| 1100° C. to about 50° C. | (5-10 min) | ambient atmosphere |

To investigate the optical properties, the densely sintered test pieces were ground plane-parallel (20-μm diamond disc) to a thickness of 2 mm±0.02 mm and a diameter of 18 mm±0.5 mm and then polished (SiC paper, grit 1000).

The densely sintered discs had a contrast ratio CR of only 78.95%. They therefore showed a very high translucence and the favourable influence of the use of vacuum and of the subsequent atmosphere exchange during the dense sintering.

### Example 7—Suspension with 83 wt.-% Zirconia

For the preparation of a suspension with 83 wt.-% zirconia and the processing thereof to form test pieces (A), Example 1 was repeated with the variation that the solution contained 164.5 g distilled water, 3.15 g dispersant containing citric acid or citric acid salt (Dolapix CE64, from Zschimmer & Schwarz) and 2.0 g tetramethylammonium hydroxide, and that 810 g zirconia powder which was partially stabilized with 4.25 mol-% Y₂O₃(TZ-PX-551 from TOSOH Corporation, primary particle size: 90 nm) were added.

The test pieces obtained according to (A) were debound at 500° C. and then had the following properties:
Pore volume: 0.11087 cm³/g
Pore radius: 0.0248 μm
Density: 3.5474 g/cm³ (test piece: length=5 mm±1 mm, width=5 mm±1 mm and height=10 mm±1 mm)

The debound test pieces were then presintered at 1050° C. for 2 h. The presintered blanks obtained then had the following properties:
Pore volume: 0.10282 cm³/g
Pore radius: 0.0315 μm
Density: 3.6838 g/cm³ (test piece: length=5 mm±1 mm, width=5 mm±1 mm and height=10 mm±1 mm)

### Example 8—Suspension with 83 wt.-% Zirconia

For the preparation of a suspension with 83 wt.-% zirconia and the processing thereof to form test pieces (A), Example 1 was repeated with the variation that the solution contained 164.5 g distilled water, 3.15 g dispersant containing citric acid or citric acid salt (Dolapix CE64, from Zschimmer & Schwarz) and 2.0 g tetramethylammonium hydroxide, and that 810 g zirconia powder which was partially stabilized with 5.0 mol.-% Y₂O₃(TZ-PX-430 from TOSOH Corporation, primary particle size: 90 nm) were added.

The test pieces obtained according to (A) were debound at 500° C. and then had the following properties:
Pore volume: 0.10559 cm³/g
Pore radius: 0.0253 μm
Density: 3.7831 g/cm³ (test piece: length=5 mm±1 mm, width=5 mm±1 mm and height=10 mm±1 mm)

The debound test pieces were then presintered at 1050° C. for 2 h. The presintered blanks obtained then had the following properties:
Pore volume: 0.10311 cm³/g
Average pore radius: 0.0318 μm
Density: 3.9083 g/cm³ (test piece: length=5 mm±1 mm, width=5 mm±1 mm and height=10 mm±1 mm)

1. Process for the preparation of a zirconia blank for the preparation of a dental restoration, in which
(a) a suspension of zirconia in a liquid medium is introduced into a mould having pores,
(b) the liquid medium is at least partially removed via the pores and (c) the blank formed is removed from the mould,
wherein the suspension has a zirconia content of from 68 to 88 wt.-%,
wherein the liquid medium comprises organic components in an amount of not more than 5 wt.-%, relative to the amount of solid in the suspension, and
wherein the blank has a density of from 3.3 to 4.0 g/cm³ and/or a pore volume of from 0.08 to 0.14 cm³/g and/or a pore diameter of from 0.02 to 0.12 μm, measured as D₅₀ value relative to the volume of the particles.

2. Process according to claim 1, in which the zirconia in the suspension has a particle size of from 50 to 250 nm, measured as the d₅₀ value and relative to the volume of the particles.

3. Process according to claim 1, in which the zirconia in the suspension has a primary particle size of from 30 to 100 nm.

4. Process according to claim 1, in which the zirconia is stabilized with Y₂O₃, La₂O₃, CeO₂, MgO and/or CaO.

5. Process according to claim 1, in which the liquid medium comprises organic components in an amount of not more than 3 wt.-%, relative to the amount of solid in the suspension, or in an amount of 0.05 to 5 wt.-%, relative to the amount of solid in the suspension.

6. Process according to claim 1, in which the liquid medium comprises at least one compound selected from amino alcohol, glycol, carboxylic acid and carboxylic acid salt.

7. Process according to claim 1, in which the suspension has a viscosity of from 5 to 500 mPas, measured at a shear rate of 0.1 to 1000 s⁻¹ and a temperature of 25° C.

8. Process according to claim 1, in which the suspension comprises a mixture of zirconia powders with different composition.

9. Process according to claim 1, in which in step (a) suspensions of zirconia powders with different composition are introduced into the mould one after another.

10. Process according to claim 1, in which the blank is a block, a block with an interface, a disc or a toothlike preform.

11. Process according to claim 1, in which the blank formed in step (c) has a holding device.

12. Process according to claim 1, in which the blank formed in step (c) is dried.

13. Process according to claim 1, in which the blank has a density of from 3.35 to 3.9 g/cm³
and/or
a pore volume of from 0.08 to 0.12 cm³/g
and/or
a pore diameter of from 0.03 to 0.10 μm, measured as D₅₀ value relative to the volume of the particles.

14. Process according to claim 1, in which the blank is presintered.

15. Process according to claim 14, in which the presintered blank has a density of from 3.4 to 4.0 g/cm³
and/or
a pore volume of from 0.08 to 0.14 cm³/g
and/or
a maximum pore diameter of less than 0.15 μm.

16. Process for the preparation of a dental restoration, in which the blank according to claim 1 is given the shape of the dental restoration and the blank is densely sintered at a sintering temperature of from 1200 to 1600° C.

17. Process according to claim 16, in which the period for heating the blank from room temperature to the sintering temperature, holding at the sintering temperature and cooling to a final temperature is not more than 120 min.

18. Process according to claim 16, in which the blank
(a) is subjected to a first heat treatment,
(b) is subjected to a second heat treatment and
(c) is cooled,
wherein the heat treatment in step (a) is effected at a lower pressure than the heat treatment in step (b).

19. Process according to claim 16, in which the dental restoration is a bridge, an inlay, an onlay, a crown, an implant, a veneer, a facet or an abutment.
